(19) United States
(12) Patent Application Publication
(10) Pub. No.: US 2007/0033717 A1
(43) Pub. Date: Feb. 15, 2007

(54) Flash memory audio strap for eyeglasses and goggles

(76) Inventor: Karen Lenore Anderson, Dixon, CA (US)

Correspondence Address: KAREN ANDERSON, 2732 QUEENSBERRY DRIVE, HUNTINGTOWN, MD, 20639, US

(21) Appl. No.: 11/158,102

(22) Filed: Aug. 12, 2005

(51) Int. Cl. A61F 9/02

(52) U.S. Cl. 2/428

(57) ABSTRACT

This is a flash memory audio optical strap with volume control and stereo ear bud speakers. This device may be used as a sunglass strap, diving mask strap, swim goggle strap or any other type of optical restraint strap. The flash memory card is removable and encased in a flexible silicone type strap.

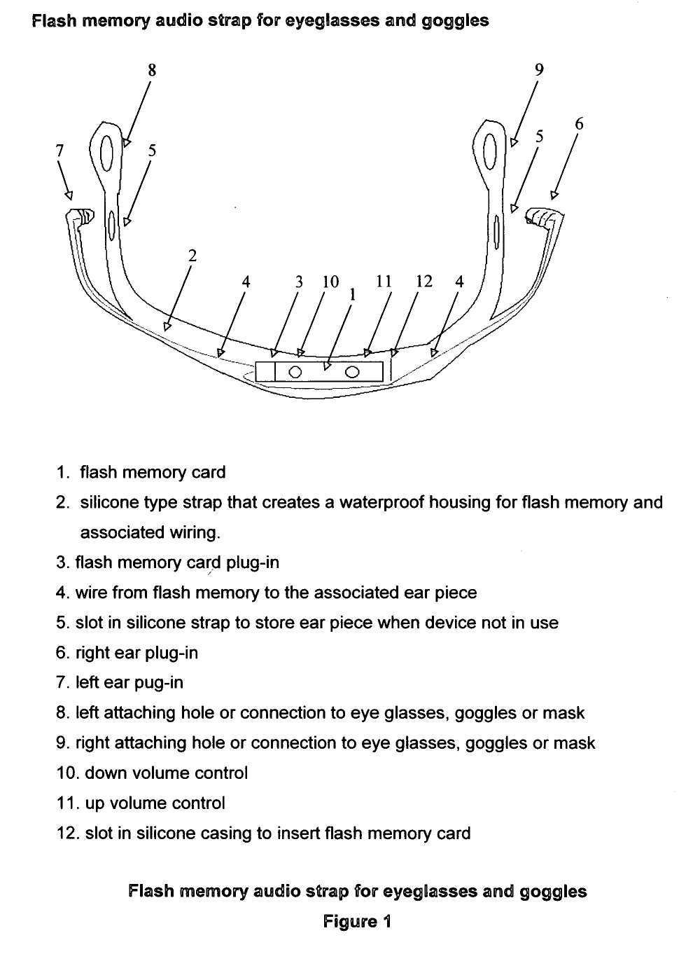

### BACKGROUND OF THE INVENTION

This device uses flash memory technology currently used for the purposes of music download and music playback. This invention utilizes this technology by encasing it in silicone housing in a light and compact strap that can attach to glasses, swim goggles, dive masks or other eyewear. It functions as a strap that keeps optical devices on the head during activities or as a way to let glasses rest around a person's neck when the glasses are not in use. It also functions as a playback audio device where digital recordings can be stored and then listened to by inserting the two earpieces in the strap into the wearer's ears.

### BRIEF SUMMARY OF THE INVENTION

This is a flash memory audio optical strap with volume control and stereo ear bud speakers. This device may be used as a sunglass strap, diving mask strap, swim goggle strap or any other type of optical restraint strap. The flash memory card is removable and encased in a flexible silicone type strap. The strap has an internal wire that extends to the ear bud speakers encased within the silicone type housing. The strap further extends to connections for swim, ski, bike goggles, dive masks and eye glasses. An up/down volume control is located on the flash memory card as well as a shuffle, play through, on/off switch. The flash memory is removable and the card is USB compatible. The entire silicone type casing is waterproof to allow the device to be submersed without affecting the playback device.

### BRIEF DESCRIPTION OF THE SEVERAL VIEWS OF THE DRAWING

The strap in the drawing is depicted without any optical device attached. The drawing is self explanatory.

1. This invention is designed to secure a flash memory audio device in a watertight strap that attaches to any optical device (glasses, goggles, mask, etc.). The strap serves two functions:
1. The strap is waterproof and allows the wearer to take the flash memory audio device into all types of weather conditions, into the water and under the water without any water damaging the flash memory audio device.
2. The strap also serves a second function as it keeps the optical device that it is attached to (glasses, goggles, mask, etc.) securely on the wearers head.
